(19) United States
(12) Patent Application Publication
(10) Pub. No.: US 2005/0159357 A1
(43) Pub. Date: Jul. 21, 2005

(54) Treatment of rheumatoid arthritis with soluble Fas-ligand cross-linkers

(75) Inventors: Vincent Jacques Hurez, Albany, CA (US); Seth G. Michelson, San Jose, CA (US); Lisl Katharine Shoda, Menlo Park, CA (US); Herbert Struemper, Bethlehem, PA (US); Leif Gustaf Wennerberg, Mountain View, CA (US)

Correspondence Address: FISH & RICHARDSON P.C., PO BOX 1022, MINNEAPOLIS, MN, 55440-1022, US

(73) Assignee: Entelos, Inc., Foster City, CA (US)

(21) Appl. No.: 11/017,211

(22) Filed: Dec. 17, 2004

Related U.S. Application Data
(60) Provisional application No. 60/530,688, filed on Dec. 17, 2003.

(51) Int. Cl. A61K 48/00; A61K 38/19; A61K 38/17

(52) U.S. Cl. 514/12; 514/44

(57) ABSTRACT

The invention encompasses novel methods of treating rheumatoid arthritis and its symptoms and novel methods of identifying and screening for drugs useful in the treatment of rheumatoid arthritis and its clinical symptoms. Targeted manipulation of a computer model of a human rheumatic joint provided the surprising result that cross-linking soluble Fas-ligand (sFasL) has a significant impact on the pathophysiology of rheumatoid arthritis. The symptoms of rheumatoid arthritis may be alleviated by administering a sFasL-specific cross-linker.

### RELATED APPLICATIONS

This application claims the benefit of U.S. Provisional Application No. 60/530,688, filed Dec. 17, 2003, which is herein incorporated by reference.

### I. INTRODUCTION

1. Field of the Invention

This invention relates to novel methods of treating rheumatoid arthritis and methods of identifying compounds useful in treating rheumatoid arthritis.

2. Background of the Invention

There are more than 100 forms of arthritis and of them, rheumatoid arthritis is the most painful and crippling form. Rheumatoid arthritis, a common disease of the joints, is an autoimmune disease that affects over 2 million Americans, with a significantly higher occurrence among women than men. In rheumatoid arthritis, the membranes or tissues (synovial membranes) lining the joints become inflamed (synovitis). Over time, the inflammation may destroy the joint tissues, leading to disability. Because rheumatoid arthritis can affect multiple organs of the body, rheumatoid arthritis is referred to as a systemic illness and is sometimes called rheumatoid disease. The onset of rheumatoid disease is usually in middle age, but frequently occurs in one's 20s and 30s. See the Merck Manual, Sixteenth Edition, section 106 for a further discussion.

The pain and whole-body (systemic) symptoms associated with rheumatoid disease can be disabling. Over time, rheumatoid arthritis can cause significant joint destruction, leading to deformity and difficulty with daily activities. It is not uncommon for people with rheumatoid arthritis to suffer from some degree of depression, which may be caused by pain and progressive disability. A study reports that one-fourth of people with rheumatoid arthritis are unable to work by 6 to 7 years after their diagnosis, and half are not able to work after 20 years (O'Dell J R (2001). Rheumatoid arthritis: The clinical picture. In W J Koopman, ed., Arthritis and Allied Conditions: A Textbook of Rheumatology, 14th ed., vol. 1, chap. 58, pp. 1153-1186. Philadelphia: Lippincott Williams and Wilkins). Musculoskeletal conditions such as rheumatoid arthritis cost the U.S. economy nearly $65 billion per year in medical care and indirect expenses such as lost wages and production. (0005] Synovial inflammation, rapid degradation of cartilage, and erosion of bone in affected joints are characteristic of rheumatoid arthritis (RA). Recent evidence indicates that skeletal tissue degradation and inflammation are regulated through overlapping but not identical processes in the rheumatoid joint and that therapeutic effects on these two aspects need not be correlated. Due to the complexity of the biological processes in the joint, mathematical and computer models can be used to help better understand the interactions between the various tissue compartments, cell types, mediators, and other factors involved in joint disease and healthy homeostasis. Several researchers have constructed simple models of the mechanical environment of the joint, rather than the biological processes of rheumatoid arthritis, and compared the results to patterns of disease and development in cartilage and bone (Wynarsky & Greenwald, J. Biomech. 16:241-251 (1983); Pollatschek & Nahir, J. Theor. Biol. 143:497-505 (1990); Beaupre et al., J. Rehabil. Res. Dev. 37:145-151 (2000); Shi et al. Acta Med. Okayama, 17:646-653 (1999)). A computer manipulable mathematical model of joint diseases that includes multiple compartments including the synovial membrane and the interactions of these compartments is described in published PCT application WO 02/097706, published 5 December 2002 and U.S. patent application Ser. No. 10/154,123, published 24 Apr. 2003 as 2003-0078759. Both publications are incorporated herein by reference in their entirety.

Rheumatoid arthritis is a chronic disease that, at present, can be controlled but not cured. The goal of treatment is relief of symptoms and keeping the disease from getting worse. Treatments for rheumatoid arthritis are designed to relieve pain, reduce inflammation, slow or stop the progression of joint damage, and improve a person's ability to function. Current approaches to treatment include lifestyle changes, medication, surgery, and routine monitoring and care. Medications used for the treatment of rheumatoid arthritis can be divided into two groups based on how they affect the progression of the disease: (1) symptom-relieving anti-rheumatic drugs and (2) disease-modifying anti-rheumatic drugs.

Medications to relieve symptoms, such as pain, stiffness, and swelling, may be used. Nonsteroidal anti-inflammatory drugs (NSAIDs), such as aspirin, ibuprofen, and naproxen are used to control pain and may help reduce inflammation. They do not control the disease or stop the disease from getting worse. Corticosteroids, such as prednisone and methylprednisolone (Medrol), are used to control pain and reduce inflammation. They may control the disease or stop the disease from getting worse; however, using corticosteroids as the only therapy for an extended time is not considered the best treatment. Corticosteroids are often used to control symptoms and flares of joint inflammation until anti-rheumatic drugs reach their full effectiveness, which can take up to 6 months. Nonprescription medications such as acetaminophen and topical medications such as capsaicin are used to control pain, but do not usually affect joint swelling or worsening of the disease.

Disease-modifying anti-rheumatic drugs (MARDs) are used to control the progression of rheumatoid arthritis and to try to prevent joint deterioration and disability. These anti-rheumatic drugs are often given in combination with other anti-rheumatic drugs or with other medications, such as nonsteroidal anti-inflammatory drugs. Disease-modifying anti-rheumatic drugs commonly prescribed for rheumatoid arthritis include antimalarial medications such as hydroxycholoroquine (Plaquenil) or chloroquine (Aralen), methotrexate (e.g., Rheumatrex), sulfasalazine (Azulfidine), leflunomide (Arava), etanercept (Enbrel), infliximab (Remicade), adalimumab (Humira), and anakinra (Kineret). DMARDs less commonly prescribed for rheumatoid arthritis include azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). Some of these anti-rheumatic drugs can take up to 6 months to work. Many have serious side effects.

Thus a need exists for new, therapeutically effective drugs for the treatment of rheumatoid arthritis as well as new methods for identifying such drugs.

### SUMMARY OF THE INVENTION

One aspect of the invention provides methods of alleviating at least one symptom of rheumatoid arthritis comprising cross-linking sFasL in a joint of a patient having rheumatoid arthritis. In a preferred embodiment, cross-linking sFasL increases macrophage apoptosis by at least 130%. In preferred embodiments, the patient is a methotrexate resistant patient, a TNF-α blockade cartilage nonresponder (CNR), a TNF-α blockade hyperplasia nonresponder (HNR), or a TNF-α blockade double nonresponder (DNR).

Another aspect of the invention provides methods of decreasing density of synovial cells in a joint comprising cross-linking sFasL in a joint of a patient having a condition associated with abnormally increased synovial cell density.

In yet another aspect, the invention provides methods of decreasing cartilage degradation in a joint comprising cross-linking sFasL in a joint of a patient having a condition associated with an abnormally high rate of cartilage degradation.

One aspect of the invention provides methods of decreasing IL-6 concentration in synovial tissue comprising cross-linking sFasL in a joint of a patient having a condition associated with an abnormally high concentration of IL-6 in synovial tissue.

Yet another aspect of the invention provides methods of alleviating at least one symptom of rheumatoid arthritis, comprising cross-linking sFasL in a joint of a patient having rheumatoid arthritis and administering an anti-rheumatic drug to the patient. The anti-rheumatic drug can be any drug that, in combination with sFasL cross-linking, provides a better clinical outcome than treatment with sFasL cross-linking or administration of the anti-rheumatic drug alone. The anti-rheumatic drug can be a symptom-relieving anti-rheumatic drug or a disease-modifying anti-rheumatic drug. Exemplary symptom-relieving anti-rheumatic drugs include aspirin, ibuprofen, naproxen, and corticosteroids, such as prednisone and methylprednisolone (Medrol). Exemplary disease-modifying anti-rheumatic drugs include hydroxycholoroquine (Plaquenil), chloroquine (Aralen), methotrexate (e.g., Rheumatrex), sulfasalazine (Azulfidine), leflunomide (Arava), etanercept (Enbrel), infliximab (Remicade), adalimumab (Humira), anakinra (Kineret), azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). In preferred embodiments, the anti-rheumatic drug is methotrexate, a TNF-α antagonist, an interleukin-1 receptor antagonist, such as Anakinra, or a steroid, such as methylprednisolone.

Yet another aspect of the invention provides methods of manufacturing a drug for use in the treatment of rheumatoid arthritis comprising (a) identifying a compound as useful in the treatment of rheumatoid arthritis and (b) formulating said compound for human consumption. The compound is identified as useful for treating rheumatoid arthritis by (i) assaying the compound for the ability to cross-link sFasL and identifying the compound as a cross-linker, (ii) comparing an amount of macrophage apoptosis in the presence of the cross-linker with an amount of macrophage apoptosis in the absence of the cross-linker, and (iii) selecting the compound as useful in the treatment of rheumatoid arthritis when the amount of macrophage apoptosis in the presence of the cross-linker is greater than the amount of macrophage apoptosis in the absence of the cross-linker.

Another aspect of the invention provides methods of identifying a compound useful in the treatment of rheumatoid arthritis, which method comprises (a) assaying the compound for the ability to cross-link sFasL and identifying the compound as a cross-linker, (b) comparing an amount of macrophage apoptosis in the presence of the cross-linker with an amount of macrophage apoptosis in the absence of the cross-linker; and (c) selecting the compound as useful in the treatment of rheumatoid arthritis when the amount of macrophage apoptosis in the presence of the cross-linker is greater than the amount of macrophage apoptosis in the absence of the cross-linker. In one embodiment, a collection of compounds may be screened by repeating steps (a), (b), and (c) for each compound in a collection of compounds, wherein at least one compound of the collection is selected as useful for the treatment of rheumatoid arthritis.

The amount of macrophage apoptosis may be determined by any apoptosis measurement technique, now known or discovered in the future. One embodiment of the invention measures the amount of macrophage apoptosis by a process comprising the steps of exposing a population of cells to an inducer of apoptosis in the presence or absence of the compound, and measuring the percentage of cells having DNA fragmentation, wherein the percentage of cells having DNA fragmentation represents the amount of macrophage apoptosis. The percentage of cells having DNA fragmentation may be measured by any method know in the art, including propidium iodide uptake or TUNEL (terminal deoxynucleotidyl transferase-mediated 2′-deoxyuridine 5′-triphosphate-biotin nick-end labeling) assay. In yet another embodiment of the invention, the amount of macrophage apoptosis is measured by a process comprising the steps of exposing a population of cells to an inducer of apoptosis in the presence or absence of the compound, and measuring the percentage of cells expressing phosphatidylserine on the extracellular surface of the cell membrane, wherein the percentage of cells expressing phosphatidylserine on the extracellular surface of the cell membrane represents the amount of macrophage apoptosis. Preferably the expression of phosphatidylserine on the extracellular surface of the cytoplasmic membrane is measured by binding of annexin V to the phosphatidylserine.

Another aspect of the invention provides packages comprising a compound capable of cross-linking soluble Fas ligand and a label with instructions for administering the compound for treating rheumatoid arthritis.

An aspect of the invention provides pharmaceutical compositions for the treatment of rheumatoid arthritis comprising a compound capable of cross-linking soluble Fas ligand and a pharmaceutical excipient.

Yet another aspect of the invention provides methods of alleviating at least one symptom of rheumatoid arthritis comprising administering a therapeutically effective amount of a compound capable of cross-linking soluble Fas ligand in combination with an anti-rheumatic drug to a patient having rheumatoid arthritis. The disease-modifying anti-rheumatic drug can be any drug that, in combination with cross-linking sFasL, provides a better clinical outcome than treatment with a compound capable of cross-linking sFasL or the anti-rheumatic drug alone. Exemplary disease-modifying anti-rheumatic drugs include hydroxycholoroquine (Plaquenil), chloroquine (Aralen), methotrexate (e.g., Rheumatrex), sulfasalazine (Azulfidine), leflunomide (Arava), etanercept (Enbrel), infliximab (Remicade), adalimumab (Humira), anakinra (Kineret), azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). In a preferred embodiment, the anti-rheumatic drug is an antagonist of FLIP (FLICE-Inhibitory Protein). Preferably, the antagonist will decrease FLIP activity by at least 25%. More preferably, the antagonist decreases FLIP activity by at least 75%. Most preferably, the antagonist decreases FLIP activity by at least 95%. The antagonist of FLIP activity may be a protein, nucleic acid or small molecule inhibitor. Preferred protein antagonists of FLIP activity include, but are not limited to oxidized low-density lipoprotein, ectopic-p53, IFN-β, PPAR ligand, E1A, and hemin. Preferred small molecule inhibitors include, but are not limited to, cyclohexamide, actinomycin D, 5-fluorouracil, doxorubicin, cisplatin, sodium butyrate, bisindolylmaleimides, H7, calphostin C, chelerythrine chloride, CDDO (triterpenoid 2-cyano-3,12-dioxoolean-1,9-dien-28-oic acid), and PS-341.

It will be appreciated by one of skill in the art that the embodiments summarized above may be used together in any suitable combination to generate additional embodiments not expressly recited above, and that such embodiments are considered to be part of the present invention

### II. BRIEF DESCRIPTION OF THE FIGURES

For a better understanding of the nature and objects of some embodiments of the invention, reference should be made to the following detailed description taken in conjunction with the accompanying drawings, in which:

FIG. 1 demonstrates the effect of cross-linking sFasL on synovial cell density in a typical rheumatoid arthritis patient.

FIG. 2 demonstrates the effect of cross-linking sFasL on the rate of cartilage degradation in a typical rheumatoid arthritis patient.

FIG. 3 demonstrates the effect of cross-linking sFasL on IL-6 in synovial tissue in a typical rheumatoid arthritis patient.

FIG. 4 demonstrates the effect of cross-linking sFasL on synovial cell density in a methotrexate resistant patient.

FIG. 5 demonstrates the effect of cross-linking sFasL on the rate of cartilage degradation in a methotrexate resistant patient.

FIG. 6 demonstrates the effect of cross-linking sFasL on IL-6 in synovial tissue in a methotrexate resistant patient.

### III. DETAILED DESCRIPTION

A. Overview

The invention encompasses novel methods of treating rheumatoid arthritis and its symptoms and novel methods of identifying and screening for drugs useful in the treatment of rheumatoid arthritis and its clinical symptoms. The inventors have made the discovery that cross-linking of soluble Fas-ligand (sFasL) has a significant impact on the pathophysiology of rheumatoid arthritis. The symptoms of rheumatoid arthritis may be alleviated by administering a compound capable of cross-linking sFasL.

B. Definitions

The term “patient” refers to any warm-blooded animal, preferably a human. Patients having rheumatoid arthritis can include, for example, patients that have been diagnosed with rheumatoid arthritis, patients that exhibit one or more of the symptoms associated with rheumatoid arthritis, or patients that are progressing towards or are at risk of developing rheumatoid arthritis.

The term “cross-linker,” as used herein, refers to a compound that covalently or non-covalently facilitates multimerization of soluble Fas ligand monomers. “Cross-linking” of soluble Fas ligand refers to the assisted multimerization of Fas ligand monomers into a complex capable of stimulating Fas-mediated apoptosis.

The term “joint,” as used herein, comprises the synovial tissue, synovial fluid, articular cartilage, bone tissues, and their cellular and extracellular composition, and the soluble mediators they contain.

The term “drug” refers to a compound of any degree of complexity that can affect a biological system, whether by known or unknown biological mechanisms, and whether or not used therapeutically. Examples of drugs include typical small molecules (molecules having molecular weights of less than 1000 daltons) of research or therapeutic interest; naturally-occurring factors such as endocrine, paracrine, or autocrine factors, antibodies, or factors interacting with cell receptors of any type; intracellular factors such as elements of intracellular signaling pathways; factors isolated from other natural sources; pesticides; herbicides; and insecticides. Drugs can also include, agents used in gene therapy such as DNA and RNA. Also, antibodies, viruses, bacteria, and bioactive agents produced by bacteria and viruses (e.g., toxins) can be considered as drugs. A response to a drug can be a consequence of, for example, drug-mediated changes in the rate of transcription or degradation of one or more species of RNA, drug-mediated changes in the rate or extent of translational or post-translational processing of one or more polypeptides, drug-mediated changes in the rate or extent of degradation of one or more proteins, drug-mediated inhibition or stimulation of action or activity of one or more proteins, and so forth. In some instances, drugs can exert their effects by interacting with a protein. For certain applications, drugs can also include, for example, compositions including more than one drug or compositions including one or more drugs and one or more excipients.

“Administering” means any of the standard methods of administering a pharmaceutical composition known to those skilled in the art. Examples include, but are not limited to intravenous, intramuscular or intraperitoneal administration.

The term “methotrexate resistant patient” refers to a rheumatoid arthritis patient who does not effectively respond to methotrexate treatment or who initially responds to methotrexate and becomes refractory over time.

As used herein, a “therapeutically effective amount” of a drug of the present invention is intended to mean that amount of the compound which will inhibit an increase in synovial cells in a rheumatic joint or decrease the rate of cartilage degradation in a rheumatic joint or decrease the concentration of IL-6 in a rheumatic joint, and cause the regression and palliation of the pain and inflammation associated with rheumatoid arthritis.

The term “abnormally increased synovial cell density,” as used herein, refers to a condition in which the synovial tissue of a joint contains a number of synovial cells that is at least ten-times higher than the number of synovial cells found in the synovial tissue of a normal, i.e., non-diseased, joint.

The term “abnormally high rate of cartilage degradation,” as used herein, refers to a detectable joint space narrowing as determined by standard radiographic measures. In a non-diseased joint narrowing is not detectable.

The term “abnormally high concentration of IL-6 in synovial tissue,” as used herein, refers to a level of IL-6 in the synovial tissue of the diseased joint that is at least 3 standard deviations higher than that found in a normal, non-diseased, joint.

The term “antagonist of FLIP activity,” as used herein, refers to the property of increasing apoptosis by impeding FLIP's inhibition of caspase-8 cleavage. The decrease in FLIP activity can be achieved either through directly interfering with FLIP's ability to inhibit apoptosis or through decreasing cellular levels of FLIP protein, thereby decreasing the amount of FLIP able to bind FADD and inhibit caspase cleavage. Inhibition need not be 100% effective in order to be antagonistic.

The term “TNF-α blockade resistant patient” refers to a rheumatoid arthritis patient who does not effective respond to TNF-α blockade or who initially responds to TNF-a blockade and becomes refractory over time.

The term “TNF-α blockade cartilage nonresponder” refers to a rheumatoid arthritis patient with low initial TNF-α activity who shows decreased synovial hyperplasia, but minimal reduction in cartilage degradation in response to TNF-α blockade.

The term “TNF-α blockade hyperplasia nonresponder” refers to a rheumatoid arthritis patient with abnormally high or resistant levels of TNF-α activity who yields improvement in cartilage degradation but little decrease in synovial hyperplasia in response to TNF-α blockade.

The term “TNF-α blockade double nonresponder” refers to a rheumatoid arthritis patient with negligible initial TNF-α activity who shows poor response in both synovial hyperplasia and cartilage degradation in response to TNF-α blockade.

C. Soluble Fas Ligand

Defective inflammatory-cell apoptosis is suggested to be a fundamental driver of rheumatoid arthritis (Pope, Nat Rev Immunol 2:527-535 (2002)). The mechanism for defective apoptosis in rheumatoid arthritis has not been clearly defined. Focusing on the extrinsic pathway of Fas-FasL signaling, a strongly effective macrophage pathway, two possibilities arise: defective extracellular signaling and defective up-regulation of intracellular anti-apoptotic molecules.

Several in vivo rheumatoid arthritis animal models—including rheumatoid arthritis synovial transplants to SCID mice—show that agonizing Fas signaling, either via cross-linking the receptor with anti-Fas antibodies or via FasL gene therapy is effective in modulating joint disease. FasL gene therapy results in increased levels of membrane-bound Fas ligand (mFasL). However, because of the wide expression of Fas receptor, serious side effects (e.g. fatal hepatotoxicity) of classical anti-Fas agonists have been reported. Currently efforts are ongoing to work around this problem (Yonehara, Cytokine Growth Factor Rev 13:393-402 (2002)), but as of yet these efforts have failed to produce a non-toxic Fas agonist.

The present invention provides a novel approach to inducing Fas-mediated apoptosis in macrophages in rheumatic patients. The invention aims at specifically restoring the anti-inflammatory feedback that FasL provides, which appears to contribute to normal resolution of inflammation. We explore the hypothesis that this mechanism is broken in the rheumatic joint by the shedding of membrane-bound FasL (mFasL) to its soluble form (sFasL), which inhibits Fas-mediated apoptosis. This process occurs via a disease-specific mechanism, with matrix metalloproteases (MMPs) cleaving mFasL to release relatively high levels of sFasL (Kayagaki, et al., J Exp Med 182:1777-1783 (1995)). Although macrophages are resistant to Fas agonists, and (non-specific) inhibition of FLIP increases apoptosis, the studies making these observations do not control for MMPs effects. Inclusion of an MMP inhibitor with the non-specific FLIP inhibitor, may have caused an even stronger pro-apoptotic effect. Thus, it seems likely that both intra- and extra-cellular mechanisms determine defective apoptosis in rheumatoid arthritis.

Increased levels of MMP-3 are correlated with increased levels of sFasL in synovial fluid from rheumatic patients. Both are correlated with disease severity. (Matsuno, et al., J Rheumatol 28:22-28 (2001)). sFasL concentrations in the synovial fluid of a rheumatic joint are ˜1 ng/ml (Nozawa, et al., Arthritis Rheum 40:1126-1129 (1997); and Hashimoto, et al., Arthritis and Rheumatism 41:657-662 (1998)). A somewhat higher concentration of 1 to 50 ng/ml of sFasL is expected in synovial tissue. Moreover, monocytes store FasL, substantial amounts of which are released in soluble form upon activation, indicating another source for sFasL in the joint (Kiener, et al., J Immunol 159:1594-1598 (1997)). Despite early reports that sFasL was pro-apoptotic (Kayagaki, et al. (1995)) subsequent work showed that at least two associated homotrimers of sFasL are necessary to induce Fas-mediated apoptosis (Holler, et al., Mol Cell Biol 23:1428-1440 (2003)).

One aspect of the present invention introduces a molecule into the rheumatic joint that binds to sFasL and causes it to multimerize sufficiently to induce Fas-mediated apoptosis; thus salvaging endogenous sFasL produced by the disease and using it for therapeutic purpose. According to a theory of the invention, only at fairly high sFasL concentrations (˜1 ng/ml), as appear likely in the synovial tissue of rheumatic joints, is sFasL cross-linking sufficient to initiate physiologically significant macrophage apoptosis and affect disease status. The invention is not bound by this theory. The requirement for such a level of sFasL suggests that side effects observed from other strategies of Fas agonism would be avoided in non-inflammatory tissues. sFasL cross-linking therapy should induce significant apoptosis only in the rheumatic joint, consequently restoring an apoptotic feedback loop broken by an RA-specific mechanism. As apoptosis increases, less matrix metalloprotease would be found in the tissues, reducing shedding of membrane-bound FasL and thus sFasL levels, thus favoring normal Fas-FasL signaling. Fas-FasL interactions have been analyzed in a mathematical model of a T-cell-tumor that simulated sFasL blocking mFasL-induced apoptosis (Webb, et al., Mathematical Biosciences 179:113-129 (2002)).

D. Identifying a Compound Useful in Treating Rheumatoid Arthritis

One aspect of the invention is a method of identifying a compound useful in the treatment of rheumatoid arthritis, which method comprises (a) identifying as a cross-linker as a compound that cross-links sFasL; (b) comparing an amount of macrophage apoptosis in the presence of the cross-linker with an amount of macrophage apoptosis in the absence of the cross-linker; and (c) selecting the compound as useful in the treatment of rheumatoid arthritis when the amount of macrophage apoptosis in the presence of the cross-linker is greater than the amount of macrophage apoptosis in the absence of the cross-linker. The dynamic processes related to the biological state of a human joint afflicted with rheumatoid arthritis involve various biological variables related to the processes involved in cartilage metabolism, tissue inflammation, and tissue hyperplasia, including the following:
macrophage population dynamics including: recruitment, activation, proliferation, apoptosis and their regulation,
T cell population dynamics including: recruitment, antigen-dependent and antigen-independent activation, proliferation, apoptosis and their regulation
Fibroblast-like synoviocyte (FLS) population dynamic including: influx in the tissue, proliferation, and apoptosis and their regulation
chondrocyte population dynamics including: proliferation and apoptosis
synthesis and regulation of a variety of proteins, including: growth factors, cytokines, chemokines, proteolytic enzymes and matrix proteins, by the different cell type represented (macrophages, FLS, T cells, chondrocytes).
expression of adhesion molecules by endothelial cells
transport of mediators between synovial tissue and cartilage
interaction between cytokines or proteases and their natural inhibitors, antigen presentation, and
binding of therapeutic agents to cellular mediators (TNF-α antagonists, such as etanercept and infliximab, and IL-1 R antagonists, such as anakinra).
   Based on observations of an in silico model providing mathematical representations of a human joint afflicted with rheumatoid arthritis, we found that cross-linking sFasL will alleviate the symptoms of rheumatoid arthritis, especially decreasing the density of synovial cells, decreasing cartilage degradation, and decreasing IL-6 concentration in synovial tissue. These observations also take into account vascular volume and the effect of therapeutic agents such as methotrexate, steroids, non-steroidal anti-inflammatory drugs, soluble TNF-α receptor, TNF-α antibody, and interleukin-1 receptor antagonists.

In silico modeling integrates relevant biological data—genomic, proteomic, and physiological—into a computer-based platform to reproduce a system's control principles. A representative model is described in co-pending U.S. patent application Ser. No. 10/154,123, published 24 Apr. 2003 as 2003-0078759. Three key clinical outcomes are of particular interest in the present model: synovial cell density, the rate of cartilage degradation and the level of IL-6 in synovial tissue. Given a set of initial conditions representing a defined disease state, these computer-based models can simulate the system's future biological behavior, a process termed biosimulation. The present invention arose from observations of these conditions. 1. Identification of sFasL Cross-Linking as an Rheumatoid Arthritis Therapy

We have discovered, based on the effects of sFasL activation of Fas-mediated apoptosis by the model described above, sFasL cross-linking is predicted to be an effective therapy for rheumatoid arthritis.

The biological effects of sFasL are still being explored. The pro-apoptotic effect of Fas cross-linking is clear in many important contexts relevant to rheumatoid arthritis. However, sFasL cross-linking can potentially have pro-inflammatory effect, an undesirable result in rheumatoid arthritis. sFasL cross-linking could stimulate: production of IL-8 by FLS (Sekine, et al., Biochem. Biophys. Res Commun 228:14-20 (1996)), proliferation of T-cells (Desbarats, et al., Proc Natl Acad Sci USA 96:8104-8109 (1999)); and production of TNFα, IL-8, and IL-10 by macrophages (Park, et al., J Immunol 170:6209-6216 (2003)).

We used two approaches in estimating the apoptotic effects of sFasL cross-linking: (A) using data of MMP inhibition on FasL/Fas-mediated apoptosis rates; and (B) estimating sFasL levels and the corresponding apoptotic effect based on (1) tissue sFasL concentration estimation in the joint, (2) in vitro dose response to sFasL cross-linking, and (3) in vitro effects of reversing sFasL inhibition of apoptosis. All of the steps of approach B present difficult estimation problems, each contributing substantial uncertainty to the final quantification. Approach A introduces one, better understood uncertainty, which is at least as well constrained by experimental data as approach B, and the assumption is easier to validate experimentally.

Estimates based on approach A are the most appropriate, specifically because of the two effects included in these experiments, which closely mimic the effects of sFasL cross-linking: (i) removal of anti-apoptotic effects of non-triggering sFasL monomer; and (ii) increased level of mFasL signaling because of reduced cleavage to sFasL. Cross-linked sFasL should yield similar apoptotic signal to that of mFasL, and cross-linking will also alleviate the blocking effects of sFasL monomer, by removing monomers from solution.

Utilizing approach A, the effect of sFasL cross-linking on monocyte/macrophage apoptosis was quantified and explicitly represented in a computer model of rheumatoid arthritis. As the effect of sFasL cross-linking on this macrophage apoptosis is not precisely quantified, a range of effects was defined in order to characterize the contribution of sFasL cross-linking (Table 1). The “lower max effect” value represents the lowest documented effect taking in consideration possible redundancies with other proteins, the “upper max effect” is the maximal effect of sFasL activity on each pathway and the “most likely max effect” is the estimation of the realistic contribution of sFasL activity in each pathway, taking in consideration the in vivo environment and potential redundancies.

TABLE 1
Effect of sFasL Cross-Linking on Joint Model

| | Lower | Most likely | Upper |
|---|---|---|---|
| Hypothesis | max effect | max effect | max effect |
| macrophage apoptosis | 0% | 15% | 68% |

There are no studies that directly implicate sFasL, in either its monomeric or multimeric form, in modulating macrophage apoptosis. However, the success of Fas agonists in animal models suggest that Fas-mediated apoptosis of macrophages can be affected by interaction between multimeric sFasL and Fas. FasL shedding has been shown to inhibit neutrophil apoptosis, suggesting that macrophages might experience a similar anti-apoptotic mechanism (LeNegrate, et al., Cell Death Differ. 10:153-162 (2003)). MMP inhibition in ovarian and cervical cancer lines expressing FasL, whose responses to Fas agonists and FLIP antagonists are similar to those of RA macrophages, results in apoptosis of 30% of cells after 24 hrs. (Knox, et al., J Immunol 170:677-685 (2003)). For the most likely results, however, 30% apoptosis in 24 hrs was considered to be too high. Correcting for the estimated concentration of sFasL in tissue of 1-50 ng/ml reduces the rather strong apoptotic effect by approximately 50%, thus the most likely maximum effect is an increase in Fas-mediated apoptosis of 15% in 24 hr.

The upper maximum effect was determined based on the results of Knox et al. (2003) and assumes that sufficient FasL signaling to completely overcome FLIP inhibition will occur. Thus the upper maximum effect on macrophage apoptosis in an increase of 68% after 24 hr. For the lower maximum effect, FLIP inhibition is assumed to prevent all Fas/FasL signaling, and thus sFasL cross-linking would have no effect.

2. sFasL Cross-Linking in Rheumatoid Arthritis Patients

The clinical impact of sFasL cross-linking on synovial cell density, cartilage degradation, and IL-6 levels was first simulated in a computer model of rheumatoid arthritis. The results of the simulation showed that administering a sFasL cross-linker for 6 months could improve the rheumatoid arthritis clinical outcome by reducing cartilage degradation by 14 to 52%, synovial cell hyperplasia by 38 to 65% and IL-6 levels in synovial tissue by 25 to 80%, assuming that cross-linking of sFasL is sufficient to overcome FLIP inhibition.

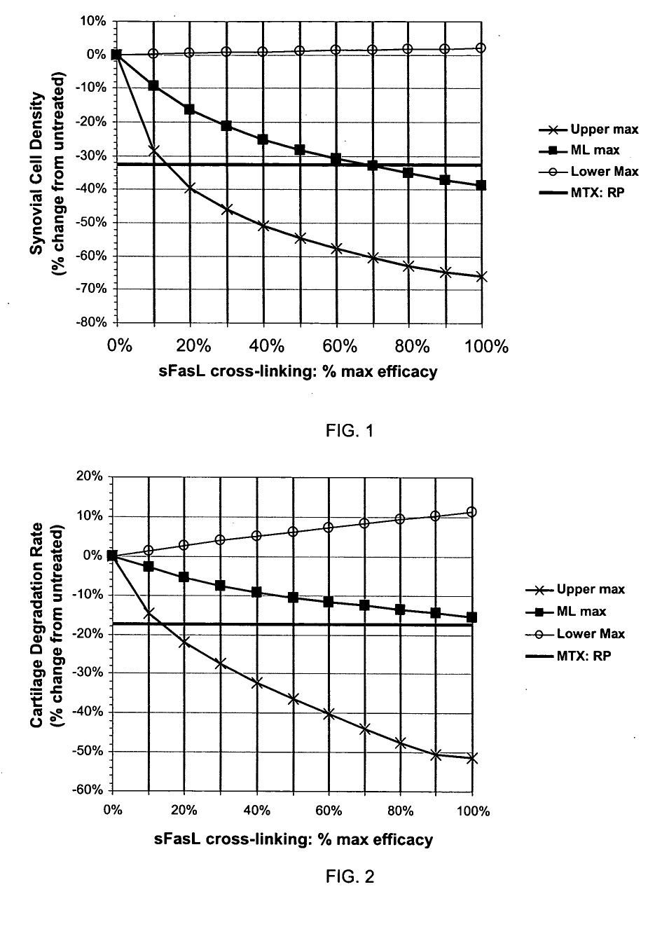

FIG. 1 demonstrates the effect of sFasL activation on synovial cell density. A decrease in synovial cell density >33% (result of methotrexate therapy) can be reached in two of the three hypothesized levels: for the upper max hypothesis, efficacy of sFasL cross-linking has to be >15% the assessed maximum; for the most likely hypothesis, efficacy of sFasL cross-linking has to be >70% of maximum, and for the lower max hypothesis, efficacy of sFasL cross-linking does not reach the MTX level. These data suggest that if sFasL can be efficiently cross-linked, clinical outcome in terms of synovial cell density should be equal to or better than methotrexate therapy.

FIG. 2 demonstrates the effect of sFasL activation on cartilage degradation rate. A decrease in cartilage degradation >17% (MTX level) can be reached in two of the three hypothesized levels: for the upper max hypothesis, efficacy of sFasL cross-linking has to be >15% of maximum; for the most likely hypothesis, efficacy of sFasL cross-linking has to ˜100% maximum; and for the lower max hypothesis, sFasL cross-linking has lower effects than MTX.

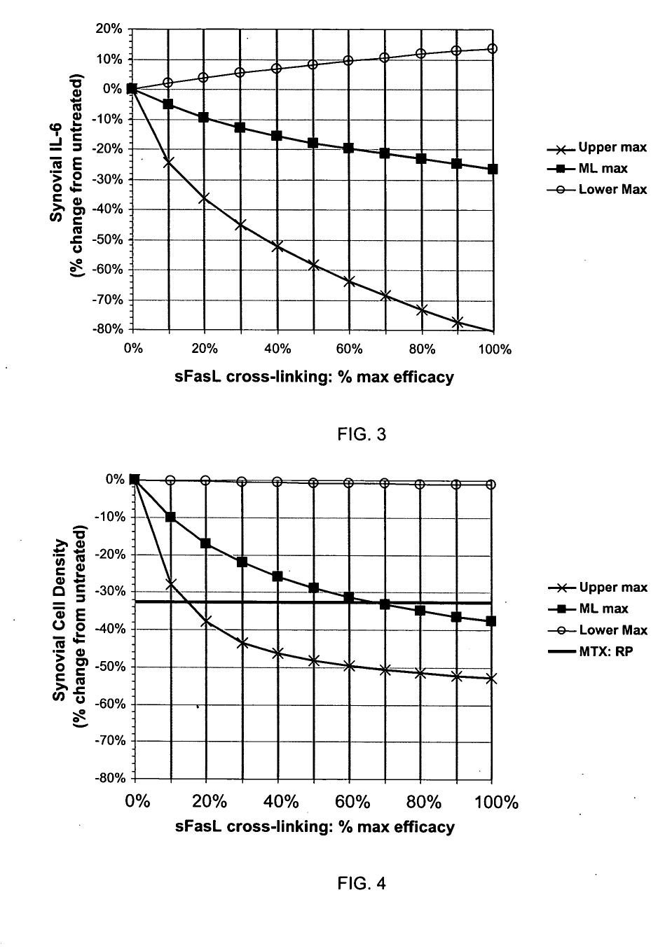

Finally, as an indirect indicator of the effect on pro-inflammatory cytokines levels in the patient's joint, synovial IL-6 was determined as a function of sFasL cross-linking. FIG. 3 demonstrates the effect of sFasL activation on IL-6 levels in synovial tissue. The level of synovial IL-6 decreased significantly (>20%) only if 10% and 60% of sFasL is cross-linked for the upper and most likely hypotheses, respectively.

3. Clinical Impact in Methotrexate Resistant Patients

A common treatment for rheumatoid arthritis is methotrexate therapy, which is known to decrease synovial cell density by approximately 30%, decrease the rate of cartilage degradation by approximately 15% and decrease the concentration of IL-6 in synovial tissue by 93%. Some rheumatoid arthritis patients do not effectively respond to methotrexate treatment (initial non-responders), while other patients who initially responded to methotrexate become refractory over time (gradual non-responders). As a group, these patients are referred to as methotrexate reduced-responders or methotrexate-resistant patients.

Simulation of sFasL cross-linking in a methotrexate resistant patient reveals a similar pattern of response to that in a non-resistant patient. The results of the simulation showed that blocking sFasL activity for 6 months in a methotrexate resistant patient could improve the rheumatoid arthritis clinical outcome by reducing synovial cell hyperplasia by 38 to 52%, cartilage degradation by 25 to 45%, and IL-6 concentration by 42 to 70%.

FIG. 4 demonstrates the effect of sFasL activation on synovial cell density in a methotrexate resistant patient. It is important to consider that MTX therapy in a methotrexate reduced-responder (i.e., methotrexate-resistant) patient results in only a 16% decrease in synovial cell density after 6 months. An effect of equivalent magnitude can be reached at much lower efficacy of sFasL cross-linking (at 7% for the upper maximum effect and 15% for the most likely maximum effect). Thus, therapy with a sFasL cross-linker is clearly more attractive than methotrexate therapy for these patients.

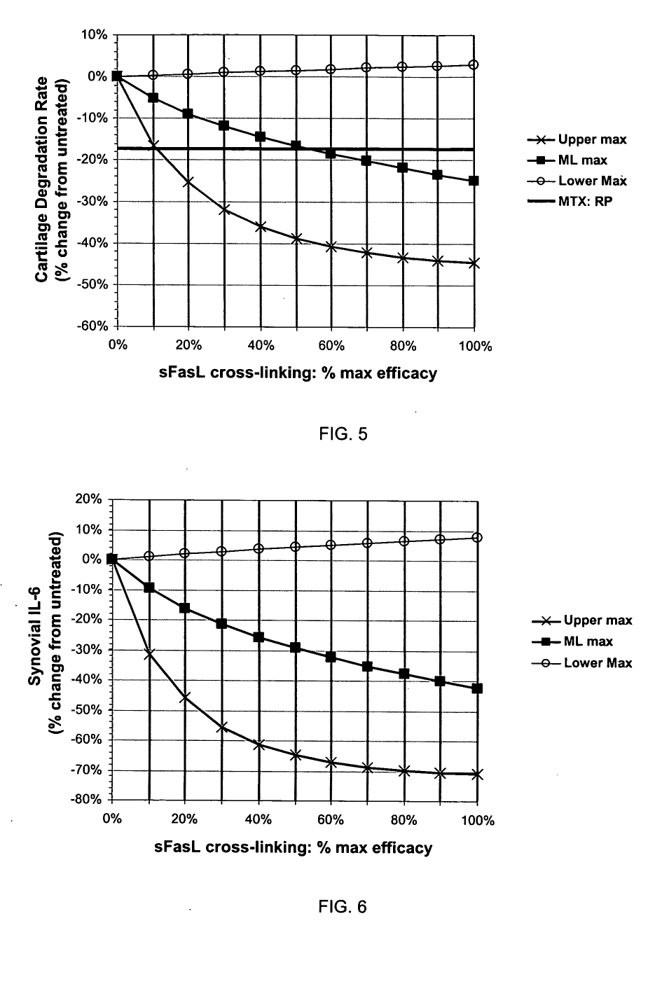

FIG. 5 demonstrates the effect of sFasL activation on cartilage degradation rate in a methotrexate resistant patient. A decrease in cartilage degradation >17% (MTX effect in reference patient) can be reached in two of the hypothesized levels: for the upper max hypothesis, efficacy of sFasL cross-linking has to be >10% of maximum; for the most likely hypothesis, efficacy of sFasL cross-linking has to be >50%. Again sFasL cross-linking provides an effect that is superior to methotrexate therapy.

FIG. 6 demonstrates the effect of sFasL activation on IL-6 concentration in a methotrexate resistant patient. The level of synovial IL-6 decreased significantly (>20%) if only 7% and 30% of sFasL is cross-linked for the upper and most likely hypothesis respectively.

Application of the in silico model of rheumatoid arthritis indicates that cross-linking sFasL represents a promising therapeutic strategy for patients suffering from rheumatoid arthritis.

E. Methods of Identifying sFasL Agonists and Anti-Rheumatic Drugs

One aspect of the invention is a method of identifying a compound useful in the treatment of rheumatoid arthritis, which method comprises (a) identifying as a cross-linker a compound that cross-links sFasL, (b) comparing an amount of macrophage apoptosis in the presence of the cross-linker with an amount of macrophage apoptosis in the absence of the cross-linker; and (c) selecting the compound as useful in the treatment of rheumatoid arthritis when the amount of macrophage apoptosis in the presence of the cross-linker is greater than the amount of macrophage apoptosis in the absence of the cross-linker.

1. Cross-Linking Assays

Compounds that cross-link, i.e., promote multimerization of, sFasL can be identified by any of a variety of methods known presently in the art or discovered in the future. Exemplary methods include, density gradient sedimentation, gel filtration chromatography, dynamic light scattering or other spectroscopic methods. In one embodiment, multimeric complexes are directly visualized by electron microscopy.

In one method, equilibrium density gradient sedimentation, continuous linear sucrose gradients (5 ml) are poured with a two-chamber gradient maker using 20% sucrose solution in hypotonic buffer and 70% sucrose solution in D₂O and kept on ice. The pH of D₂O can be adjusted to a physiologically relevant pH by dropwise addition of 10 mM NaOH. Gradients are then overlaid with 0.5 ml of sFasL in the presence of the potential cross-linker and centrifuged at 35,000 rpm at 4° C. Gradients can be fractionated by puncturing the bottom of the tube and collecting a series of fractions. The density is calculated by weighing an aliquot of each fraction.

Alternatively, sedimentation velocity experiments may be used to determine the extent of multimerization of the sFasL. In such experiments, continuous gradients are poured as described above, using 5 and 20% sucrose solutions. The equilibrium density fraction containing the peak of the sFasL loaded on the 5 to 20% continuous sucrose gradient, and centrifuged at 23,000 rpm for 1 h at 4° C. in a Beckman SW55 rotor. Fractions are collected by puncturing the bottom of the tube, and the density was measured by weighing 100 μl of each fraction. The S value may be calculated by the method of McEwen, Anal. Biochem. 20:114-149 (1967).

In yet another method, in cases where the potential sFasL cross-linker non-covalently interacts with sFasL, a non-specific covalent protein cross-linker can be used to stabilize multimeric sFasL complexes for the purposes of determining whether a test compound enhances sFasL multimerization. Most non-specific protein cross-linking reagents have two reactive groups connected by a flexible spacer arm. These reagents may have the same reactive groups at both ends (i.e., homo-bifunctional cross-linkers) or different reactive groups at the ends (i.e., hetero-bifunctional cross-linkers).

Any of a variety of well-known non-specific protein cross-linkers, such as N-hydroxysuccinimide esters (which react with primary amines) can be used. Exemplary non-specific protein cross-linkers include disuccinimidyl glutarate, disuccinimidyl suberate, bis(sulfosuccinimidyl) suberate and tris-succinimidyl aminotriacetate (a trifinctional cross-linker). After non-specific covalent cross-linking the molecular weight of the complexes can be determined by a variety of methods, including, for example, gel filtration chromatography or SDS-polyacrylamide gel electrophoresis.

2. Macrophage Apoptosis Assays

As described above, increasing macrophage apoptosis is the major contributor to the expected benefits of sFasL cross-linking. Apoptosis measurement can vary depending on the cell type and the assay used. It may be advantageous to use a combination of standard apoptotic assays (e.g., Annexin V or TUNEL assays) to measure the percentage of apoptotic monocytes/macrophages and a quantitative anti-histone ELISA to measure the global effect of sFasL activation on apoptosis.

Loss of DNA integrity is one characteristic of apoptosis. When DNA extracted from apoptotic cells is analyzed using gel electrophoresis, a characteristic “ladder” of DNA fragments is seen. However, extraction of DNA from cells is a time consuming process and alternative methods are equally suitable for detecting the characteristic fragmentation of DNA in apoptotic cells. DNA fragmentation can be detected by a variety of assay including propidium iodide assays, acridine orange/ethidium bromide double staining, TUNEL and ISNT techniques, and the assays of DNA sensitivity to denaturation.

Externalization of phosphatidylserine (PS) and phosphatidylethanolamine is yet another hallmark of apoptosis. Annexin V is a 35-36 kDa Ca²⁺-dependent, phospholipid binding protein that has a high affinity for PS and binds to cells with exposed PS. Annexin V may be conjugated to any of a variety of markers to permit it to be detected by microscopy or flow cytometry. For use in methods of identifying compounds the inhibit sFasL activity or methods of screening for compounds that inhibit sFasL activity, it is preferable to use fluorescently labeled annexin V detected by flow cytometry.

Monocytes or macrophages can be isolated from synovial fluid or peripheral blood mononuclear cells from rheumatoid arthritis patients or healthy donors by either Percoll or Histopaque (Sigma Chemical Co.) gradient centrifugation or countercurrent centrifugal elutriation (Beckman-Coulter). Monocytes can be differentiated into macrophages with RPMI containing 20% heat-inactivated fetal bovine serum (FBS) plus 1 μg/ml polymyxin B sulfate (Sigma Chemical Co.) in 24-well plates (Costar). Cells are incubated with the test compound for one to 24 hours, optionally in the presence of a DR-dependent inducer of apoptosis. The number of cells committed to apoptosis is determined by staining with labeled annexin V and a vital dye, such as propidium iodide (PI) or 7-amino-actinomycin D (7-AAD). Because externalization of PS occurs in the earlier stages of apoptosis, annexin V staining precedes the loss of membrane integrity which accompanies the latest stages of cell death resulting from either apoptotic or necrotic processes. Therefore, staining with annexin V in conjunction with vital dyes such as propidium iodide (PI) or 7-amino-actinomycin D (7-AAD) permits identification of early apoptotic cells (annexin V-positive and vital dye-negative).

F. Methods of Treatment

1. Treating Rheumatoid Arthritis with sFasL Cross-Linkers

Another aspect of this invention provides methods for alleviating at least one symptom of rheumatoid arthritis comprising administering a therapeutically effective amount of a sFasL cross-linker to a patient having rheumatoid arthritis. The sFasL cross-linker may be a protein, nucleic acid or small molecule inhibitor and may interact with sFasL either covalently or non-covalently. A preferred protein cross-linker is a non-blocking antibody, more preferably a monoclonal antibody. A non-blocking antibody of the invention will enhance multimerization of sFasL but will not interfere with binding to and resultant activation of Fas receptors on the cell surface. In a preferred embodiment, the non-blocking antibody binds specifically to a sequence from the N-terminal portion of sFasL. More preferably, the antibody will specifically bind within the N-terminal-most 15 amino acids of human sFasL, which have the sequence SLEKQIGHPSPPPEK (SEQ ID NO: 1). The invention also encompasses methods of decreasing synovial cell density, methods of decreasing cartilage degradation and methods of decreasing IL-6 concentration in synovial tissue by administering a therapeutically effective amount of a sFasL cross-linker.

A compound useful in this invention is administered to a rheumatoid arthritis patient in a therapeutically effective dose by a medically acceptable route of administration such as orally, parenterally (e.g., intramuscularly, intravenously, subcutaneously, intraperitoneally), transdermally, rectally, by inhalation and the like. The dosage range adopted will depend on the route of administration and on the age, weight and condition of the patient being treated.

Various delivery systems are known and can be used to administer a composition of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into a rheumatic joint by any suitable route. Pulmonary administration can also be employed, e.g., by use of an inhaler or nebulizer, and formulation with an aerosolizing agent. 2. Combination Therapy with sFasL Cross-Linkers and Anti-Rheumatic Drugs

In one aspect, the invention provides methods of alleviating at least one symptom of rheumatoid arthritis, comprising administering a therapeutically effective amount of a compound capable of cross-linking soluble Fas ligand in combination with an anti-rheumatic drug to a patient having rheumatoid arthritis. Preferably, the anti-inflammatory drug is selected from the group of methotrexate, a TNF-α antagonist, an interleukin-1 receptor antagonist and a steroid. More preferably, the anti-inflammatory drug is methotrexate, Etanercept, Anakinra or prednisone. In one embodiment of the invention, the patient is resistant to methotrexate or to TNF-α blockade.

Various treatment protocols were simulated alone, or in combination with cross-linking sFasL. The effects of several therapies are represented in the model. The model reproduces the impact of treatment with (1) non-steroidal anti-inflammatory drugs (NSAIDs; e.g., indomethacin), (2) Etanercept, a soluble type II TNF-a receptor, (3) methotrexate (MTX), (4) glucocorticoids (e.g., methylprednisolone), and (5) Anakinra, an IL-1 receptor antagonist (IL-1Ra).

Each therapy is implemented based on its mode of action, molecular activity, and pharmacokinetic properties as well as its recommended clinical dosing regimen. To determine the importance of time-dependent variation in drug exposure associated with the clinically recommended periodic drug administration, we compared simulation results based on the clinical schedule with results for a constant-concentration continuous dose with an equivalent serum area-under-the-curve (AUC) net drug exposure. Simulation results for the two different administration schedules differed only minimally. In order to simplify presentation of results by eliminating transient effects due to periodic administration, results discussed herein are based on continuous dose therapy simulations.

The impact of the simulated treatments results from the implemented molecular activity. For example, Etanercept is modeled as binding and neutralizing TNF-α; any subsequent changes in hyperplasia, cartilage degradation, or other measurements are a secondary consequence of this reduction in free, active TNF-α, rather than a direct or specified effect of Etanercept. The effects directly implemented for each therapy are as follows:

The primary, common mode of action of NSAIDs is the inhibition of the cyclo-oxygenase (COX) pathways and synthesis of their downstream products, especially prostaglandin-E2 (PGE2). The model implementation of NSAIDs is based on in vitro data on the dose-dependent inhibition by NSAIDs of PGE2 synthesis in macrophages, FLS, and chondrocytes. Simulation results presented are for a constant continuous dose with serum AUC drug exposure equivalent to that achieved with a dosing schedule of 50 mg indomethacin, administered orally 3 times a day.

Etanercept (exogenous sTNF-RII) is modeled as binding and neutralizing soluble TNF-α. The net binding rate of soluble receptors to TNF-α is calculated as the difference between the binding and dissociation rates as follows:

$$\frac{ⅆ}{ⅆ t} [ TNF α : sTNFR ] = {k}_{on} [ TNF α ] [ sTNFR ] - {k}_{off} [ TNF α : sTNFR ] \qquad ( eq . 1 )$$

where kₒₙ=constant of association between sTNF-R and TNF-α
k_{off}=constant of dissociation between sTNF-R and TNF-α
[TNFα]=concentration of free TNF-α
[sTNFR]=concentration of free soluble TNF-R
[TNFα:sTNFR]=concentration of bound complexes

Simulation results presented are for a constant continuous dose of Etanercept with serum AUC drug exposure equivalent to that achieved with a dosing schedule of 25 mg, administered subcutaneously twice a week.

Methotrexate therapy is implemented based on in vitro data that quantify its direct effects on particular cellular functions, including dose-dependent inhibition of T cell and FLS proliferation, mediator synthesis, and apoptosis. In addition, to account for the inhibitory effect of methotrexate on vascular proliferation and vascularization, a reduction in total endothelial adhesion molecules expression is also implemented. Simulation results presented are for a constant continuous dose with serum AUC drug exposure equivalent to a multiple of a dosing schedule of 12.5 mg/week, administered orally to account for long-lived, active metabolites of methotrexate.

Methylprednisolone is represented by the dose-dependent modulation of various cellular mediator synthesis rates according to in vitro data. Effects on other cell functions are not directly modeled but may arise from altered mediator-dependent regulation. Simulation results presented are for a constant continuous dose with serum AUC drug exposure equivalent to that of a dosing schedule of 5 mg methylprednisolone, administered orally once a day.

Anakinra, like endogenous IL-1Ra, is modeled as reducing the impact of IL-1β on all cellular functions. This is implemented by calculating an “effective” IL-1β concentration that has been adjusted to account for the impact of reduced receptor binding in the presence of the instantaneous concentration of receptor antagonist. Simulation results presented are for a constant continuous dose with serum AUC drug exposure equivalent to that of a dosing schedule of 100 mg Anakinra, administered subcutaneously once a day.

Simulation of the effect of treatment on the progression of rheumatoid disease in a virtual patient was conducted by simulating rheumatoid arthritis in the virtual patient for one year without treatment to establish a baseline in the model. Then either no treatment, a current treatment protocol or a current protocol in combination with cross-linking sFasL was modeled. sFasL cross-linking was modeled assuming (i) the “upper max effect,” which represents maximal expected effect of sFasL cross-linking on each biological process (ii) the “most likely max effect,” which is the estimation of the realistic contribution of sFasL cross-linking, taking into consideration the in vivo environment and redundancies; and (iii) the “lower max effect,” which represents the lowest documented effect taking in consideration possible redundancies with other proteins. Simulation of the “lower max effect” of cross-linking sFasL showed no beneficial effect when combined with the current treatment protocols. The effects of the simulated treatment (or lack of treatment) in a typical patient for six months on synovial cell density and cartilage degradation rate for the “upper max effect” and the “most likely max effect” are shown in TABLE 3.

TABLE 3
Effects of sFasL Cross-linking in Combination with Other Therapies

| | | | | MTX | | TNF | |
|---|---|---|---|---|---|---|---|
| | | Reference | | resistant | | non- | |
| | | patient | | patient | | responder | |
| First agent | Second agent | s.c.d. | c.d.r. | s.c.d. | c.d.r. | s.c.d. | c.d.r. |
| None | None | 100 | 100 | 100 | 100 | 100 | 100 |
| | sFasL cross-linker (most likely max effect) | 61 | 85 | 62 | 75 | 61 | 85 |
| | sFasL cross-linker (upper max effect) | 34 | 49 | 47 | 56 | 35 | 47 |
| NSAID | None | 103 | 105 | 105 | 106 | 104 | 106 |
| | sFasL cross-linker (most likely max effect) | 61 | 86 | 67 | 78 | 61 | 87 |
| | sFasL cross-linker (upper max effect) | 34 | 49 | 50 | 59 | 35 | 47 |
| Methotrexate | None | 67 | 82 | 81 | 87 | 70 | 83 |
| | sFasL cross-linker (most likely max effect) | 50 | 74 | 58 | 68 | 53 | 77 |
| | sFasL cross-linker α (upper max effect) | 30 | 42 | 42 | 44 | 30 | 39 |
| Etanercept | None | 51 | 67 | 71 | 81 | 88 | 76 |
| | sFasL cross-linker (most likely max effect) | 43 | 60 | 58 | 70 | 50 | 62 |
| | sFasL cross-linker (upper max effect) | 33 | 47 | 48 | 56 | 32 | 45 |
| Anakinra | None | 82 | 55 | 90 | 54 | 90 | 60 |
| | sFasL cross-linker (most likely max effect) | 50 | 41 | 53 | 37 | 54 | 45 |
| | sFasL cross-linker (upper max effect) | 30 | 23 | 44 | 29 | 30 | 22 |
| Steroid | None | 59 | 59 | 70 | 64 | 61 | 58 |
| | sFasL cross-linker (most likely max effect) | 45 | 52 | 54 | 51 | 47 | 53 |
| | sFasL cross-linker (upper max effect) | 30 | 33 | 43 | 40 | 30 | 31 |

s.c.d. = % of synovial cell density as compared to untreated patient
c.d.r. = % of cartilage degradation rate as compared to untreated patient

The results of the simulation in a typical rheumatoid arthritis patient showed that cross-linking sFasL in addition to administering an interleukin-1 receptor antagonist, such as Anakinra, can improve the rheumatoid arthritis clinical outcome by reducing cartilage degradation by 59 to 77% and synovial cell hyperplasia by 50 to 70%. Similarly, treatment with sFasL cross-linking in combination with administration of methotrexate, Etanercept or a steroid, such as methylprednisolone, shows decreases in synovial cell hyperplasia and cartilage degradation that cannot be achieved with the monotherapy.

Simulation of sFasL cross-linking in combination with standard anti-rheumatic treatments in a methotrexate resistant patient revealed a pattern of response similar to that in a normal methotrexate-responsive patient. The effects of the simulated treatment (or lack of treatment) in a methotrexate resistant patient for six months on synovial cell density is summarized in Table 3. The results of the simulation showed that cross-linking sFasL in addition to administering an interleukin-1 receptor antagonist, such as Anakinra, can improve the rheumatoid arthritis clinical outcome by reducing cartilage degradation by 63 to 71% and synovial cell hyperplasia by 47 to 56%. Interestingly, a combination therapy comprising sFasL cross-linking and administration of methotrexate to a methotrexate resistant patient can improve the rheumatoid arthritis clinical outcome by reducing cartilage degradation and synovial cell hyperplasia to a greater extent than achieved by sFasL cross-linking or methotrexate treatment alone. As with a typical rheumatoid arthritis patient, treatment with a compound capable of cross-linking sFasL in combination with Etanercept or a steroid, such as methylprednisolone, shows decreases in synovial cell hyperplasia and cartilage degradation that cannot be achieved with the monotherapy

TNF-α neutralizing therapies have become increasingly important in treating rheumatoid arthritis patients. However, roughly a third of all rheumatoid arthritis patients fail to achieve a clinically significant response to TNF-α neutralizing therapies. Three potential classes of TNF-α blockade resistant patients were defined in the model described above. Synovial hyperplasia and cartilage degradation are differentially affected when TNF-α varies within different ranges, leading to the identification of three nonresponder classes within the current model. Specifically, patients with low initial TNF-α activity show decreased synovial hyperplasia, but minimal reduction in cartilage degradation in response to TNF-α blockade (cartilage nonresponders, or CNRs), while patients with negligible initial TNF-α activity show poor response in both synovial hyperplasia and cartilage degradation (double nonresponders or DNRs). Alternatively, insufficient neutralization of TNF-α in patients with abnormally high or resistant levels of TNF-α activity yields improvement in cartilage degradation but poor response in hyperplasia (hyperplasia nonresponders or HNRs). Mechanistically, in patients with low levels of TNF-α, rheumatoid disease was perpetuated by increased activity of alternate macrophage activating pathways (e.g., CD40-ligation), reduced activity of anti-inflammatory cytokines (e.g., IL-10), and increased activity of degradation-promoting cytokines (e.g., IL-1β). Non-responding patients also showed altered responses to other therapies such as IL-1Ra (data not shown).

Patients who fail to achieve a significant clinical response to TNF-α blockade represent a sizable subset of the rheumatoid arthritis population. Simulation of sFasL cross-linking in combination with standard anti-rheumatic treatments in a TNF-α hyperplasia nonresponder revealed a slightly different pattern of response than in a normal TNF-α-responsive patient. The effects of the simulated treatment (or lack of treatment) in a TNF-α hyperplasia nonresponder for six months on synovial cell density and cartilage degradation is shown in Table 3. The results of the simulation showed that combination therapy comprising sFasL cross-linking and administration of methotrexate, IL-1Ra or steroid to a TNF-α blockade resistant patient showed similar improvement in clinical outcome as compared a normal TNF-α blockade responsive individual receiving the combination therapy. However, combination of sFasL cross-linking with Etanercept treatment in a TNF-α blockade resistance patient results in substantially greater decrease in synovial cell hyperplasia and lower cartilage degradation rates as compared to the monotherapy alone.

In especially preferred embodiments of the invention, the anti-rheumatic drug is an antagonist of FLIP activity. As discussed above macrophage apoptosis is believed to be a fundamental driver of rheumatoid arthritis. FLIP (FLICE-Inhibitory Protein) inhibits apoptosis induced by Fas. Thus, in some situations, even if Fas is activated by a cross-linked sFasL complex, FLIP activity may inhibit apoptosis of the target cell. Therefore, one aspect of the present invention provides methods of alleviating at least one symptom of rheumatoid arthritis comprising administering a therapeutically effective amount of a compound capable of cross-linking soluble Fas ligand in combination with an antagonist of FLIP activity to a patient having rheumatoid arthritis. Antagonists of FLIP activity and methods of identifying such antagonists are discussed in detail in co-pending U.S. patent application Ser. No. 10/980,145, filed Nov. 1, 2004, which is incorporated by reference herein.

Preferably, the antagonist will decrease FLIP activity by at least 25%. More preferably, the antagonist decreases FLIP activity by at least 75%. Most preferably, the antagonist decreases FLIP activity by at least 95%. The antagonist of FLIP activity may be a protein, nucleic acid or small molecule inhibitor. Preferred protein antagonists of FLIP activity include, but are not limited to oxidized low-density lipoprotein, ectopic-p53, IFN-β, PPAR ligand, E1A, and hemin. Preferably a nucleic acid antagonist will be an antisense inhibitor. A preferred antisense inhibitor of FLIP activity comprises the sequence, 5′-GACTTCAGCAGACATCCTAC-3′ (SEQ ID NO: 2). Preferred small molecule inhibitors of FLIP activity include, but are not limited to, cyclohexamide, actinomycin D, 5-fluorouracil, doxorubicin, cisplatin, sodium butyrate, bisindolylmaleimides, H7, calphostin C, and chelerythrine chloride.

A compound capable of cross-linking sFasL and another anti-rheumatoid drug are administered concurrently. “Concurrent administration” and “concurrently administering” as used herein includes administering a compound capable of cross-linking sFasL and another anti-rheumatoid drug in admixture, such as, for example, in a pharmaceutical composition or in solution, or as separate compounds, such as, for example, separate pharmaceutical compositions or solutions administered consecutively, simultaneously, or at different times but not so distant in time such that the compound capable of cross-linking sFasL and the other anti-rheumatoid drug cannot interact.

Regardless of the route of administration selected, the compound capable of cross-linking sFasL and other anti-rheumatoid drug are formulated into pharmaceutically acceptable unit dosage forms by conventional methods known to the pharmaceutical art. An effective but nontoxic quantity of the compound capable of cross-linking sFasL and the other anti-rheumatoid drug are employed in the treatment. The compound capable of cross-linking sFasL and other anti-rheumatoid drug may be concurrently administered enterally and/or parenterally in admixture or separately. Parenteral administration includes subcutaneous, intramuscular, intradermal, intravenous, injection directly into the joint and other administrative methods known in the art. Enteral administration includes tablets, sustained release tablets, enteric coated tablets, capsules, sustained release capsules, enteric coated capsules, pills, powders, granules, solutions, and the like.

G. Pharmaceutical Compositions

1. Antibodies

Antibodies of the invention include, but are not limited to, polyclonal, monoclonal, bispecific, human, humanized or chimeric antibodies, single chain antibodies, sFvs fragments, F(ab′) fragments, fragments produced by a Fab expression library, anti-idiotypic (anti-Id) antibodies, and epitope-binding fragments of any of the above which immunospecifically bind to sFasL. In a preferred embodiment, the antibody binds specifically to a sequence within the N-terminal portion of sFasL. More preferably, the antibody will specifically bind within the first 15 amino acids of human sFasL, which have the sequence SLEKQIGHPSPPPEK (SEQ ID NO: 1). The term “antibody” as used herein refers to molecule comprising immunoglobulins and immunologically active portions of immunoglobulins, i.e., molecules that contain an antigen binding site which immunospecifically binds sFasL. The immunoglobulins of the invention can be of any type (e.g., IgG, IgE, IgM, IgD and IgA), class, or subclass of immunoglobulin.

Monoclonal antibodies which may be used in the methods of the invention are homogeneous populations of antibodies to a particular antigen (e.g., sFasL). For the purposes of this invention a “monoclonal antibody” is an antibody produced by a hybridoma cell. Methods of making monoclonal antibody-synthesizing hybridoma cells are well known to those skilled in the art, e.g., by the fusion of an antibody producing B lymphocyte with an immortalized B-lymphocyte cell line. Preferably the monoclonal antibody will be a murine monoclonal antibody, a chimeric monoclonal antibody, a humanized monoclonal antibody, or, most preferably, a human monoclonal antibody.

A monoclonal antibody (mAb) to sFasL can be prepared by using any technique known in the art which provides for the production of antibody molecules by continuous cell lines in culture. These include but are not limited to, the hybridoma technique originally described by Kohler and Milstein (Nature 256:495-497 (1975)), the more recent human B cell hybridoma technique (Kozbor et al., Immunology Today 4:72 (1983)), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA and, IgD and any subclass thereof. The hybridoma producing the mAbs of use in this invention may be cultivated in vitro or in vivo.

The monoclonal antibodies that may be used in the methods of the invention include, but are not limited to, human monoclonal antibodies or chimeric human-mouse (or other species) monoclonal antibodies. Human monoclonal antibodies may be made by any of numerous techniques known in the art (e.g., Teng et al., 1983, Proc. Natl. Acad. Sci. U.S.A. 80, 7308-7312; Kozbor et al., 1983, Immunology Today 4, 72-79; and Olsson et al., 1982, Meth. Enzymol. 92, 3-16).

The invention provides for the use of functionally active fragments, derivatives or analogs of antibodies which immunospecifically bind to sFasL. Functionally active means that the fragment, derivative or analog is able to elicit anti-anti-idiotype antibodies that recognize the same antigen that the antibody from which the fragment, derivative or analog is derived recognized. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIAcore assay)

Other embodiments of the invention include fragments of the antibodies of the invention such as, but not limited to, F(ab′)₂ fragments, which contain the variable region, the light chain constant region and the CH1 domain of the heavy chain can be produced by pepsin digestion of the antibody molecule, and Fab fragments, which can be generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine monoclonal and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060; each of which is incorporated herein by reference in its entirety.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806; each of which is incorporated herein by reference in its entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

2. Formulation

An aspect of the invention provides methods of manufacturing a drug useful for treating rheumatoid arthritis in a warm-blooded animal. The drug is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the invention are found by reference to the 18^{th} or 19^{th} Edition of Remington's Pharmaceutical Sciences, published by the Mack Publishing Co., Easton, Pa. 18040. The pertinent portions are incorporated herein by reference.

Unit doses or multiple dose forms are contemplated, each offering advantages in certain clinical settings. The unit dose would contain a predetermined quantity of a sFasL cross-linker calculated to produce the desired effect(s) in the setting of treating rheumatoid arthritis. The multiple dose form may be particularly useful when multiples of single doses, or fractional doses, are required to achieve the desired ends. Either of these dosing forms may have specifications that are dictated by or directly dependent upon the unique characteristic of the particular compound, the particular therapeutic effect to be achieved, and any limitations inherent in the art of preparing the particular compound for treatment of cancer.

A unit dose will contain a therapeutically effective amount sufficient to treat rheumatoid arthritis in a subject and may contain from about 1.0 to 1000 mg of compound, for example about 50 to 500 mg.

In a preferred embodiment, the drug of the invention is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The drug of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The compound will preferably be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition.

The form may deliver a compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. Preferred compositions according to the current invention are prepared so that an oral dosage unit form contains between about 5.0 to about 50% by weight (%w) in dosage units weighing between 5 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

The drug of the invention may be administered parenterally, e.g., intravenously, intramuscularly, intravenously, subcutaneously, or intraperitoneally. In a preferred aspect, the drug of the invention will be administered directly to the rheumatic joint. The carrier or excipient or excipient mixture can be a solvent or a dispersive medium containing for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In a specific embodiment, it may be desirable to administer the compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the composition can be delivered in a controlled release, or sustained release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used in a controlled release system (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target (e.g., the brain, kidney, stomach, pancreas, and lung), thus requiring only a fraction of the systemic dose (see, e.g., Goodson, in Medical Applications of Controlled Release, supra, vol. 2, pp. 115-138 (1984)). Other controlled release systems are discussed in the review by Langer (1990).

In a specific embodiment where the drug of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The invention also encompasses pharmaceutical compositions comprising a compound identified by a method of the invention contained in a container and labeled with instructions for use of the composition in the treatment of rheumatoid arthritis. The kit can further comprise instructions for using dosage. Accordingly, the invention contemplates an article of manufacture comprising packaging material and, contained within the packaging material, a sFasL cross-linker, wherein the packaging material comprises a label or package insert indicating that the cross-linker modulates Fas-mediated apoptosis and can be used for treating the symptoms of rheumatoid arthritis.

### IV. EXAMPLES

The following examples are provided as a guide for a practitioner of ordinary skill in the art. The examples should not be construed as limiting the invention, as the examples merely provide specific methodology useful in understanding and practicing an embodiment of the invention.

### A. Example 1

### Cross-Linking of sFasL

Purified sFasL is combined with the test compound. The sFasL/test compound complexes are stabilized by incubation with a 10-fold molar excess of disuccinimidyl glutarate (available from Pierce Biotechnology, Inc., Rockford, Ill.) for 30 minutes at room temperature. The cross-linking reaction is quenched by addition of 20 mM (final concentration) glycine. The resulting complexes are mixed with glycerin and sprayed on freshly prepared mica. The probe is dried at 10⁻⁵ atm for 4 hr and rotary shadowing is performed by vaporizing platinum as described in Engel, Methods Enzymol. 245:496-488 (1994). After stabilization by a coat of vaporized coal (900), the replica is detached from the mica support at the water-air interface, fixed on a grid, dried and analyzed by electron microscopy. Multimeric complexes are visible at a magnification of ×150,000.

### B. Example 2

### Apoptosis Activation and Annexin V Assay

Nine-day-adherent RA SF macrophages are combined with recombinant sFasL (1-50 ng/ml) and apoptosis is induced by adding sFasL cross-linker and incubating for 24 hours. Cells are washed twice with cold PBS and then resuspended in 10 mM HEPES, pH 7.4; 140 mM NaCl; 2.5 mM CaCl₂ at a concentration of ˜1×10⁶ cells/ml. 100 μl of the solution (−1×10⁵ cells) is transferred to a 5 ml culture tube. 5 μl of 2.5 μg Annexin V-phycoerythrin and 2.5 μg vital dye 7-AAD are added to each tube, gently mixed and incubated at room temperature in the dark for 15 minutes. 400 μl phosphate buffered saline (PBS) is added to each tube and the cells are analyzed by cell cytometry as soon as possible (within one hour). The percentage of apoptotic cells is measured by the percentage of Annexin V positive cells.

### C. Example 3

### TUNEL Assay

Nine-day-adherent RA SF macrophages are combined with recombinant sFasL (1-50 ng/ml) and apoptosis is induced by adding sFasL cross-linker and incubating for 24 hours. The cultures are centrifuged at 400× G for minutes, the supernatant is discarded and the cells are resuspended in 0.5 ml phosphate buffered saline (PBS). The cells are fixed by adding the cell suspension to 5 ml of 1% (w/v) paraformaldehyde in PBS, placing it on ice for 15 min, washing the cells twice in PBS twice, and finally combining the cells suspended in 0.5 ml PBS with 5 ml ice-cold 70% (v/v) ethanol. The cells stand for a minimum of 30 minutes on ice or in the freezer before proceeding to the staining step.

The tubes are swirled to resuspend the cells and 1.0 ml aliquots of the cell suspensions (˜2-4×10⁵ cells/ml) are removed and placed in 12×75 mm centrifuge tubes. The cell suspensions are centrifuged for 5 min at 300× g and the 70% (v/v) ethanol removed by aspiration. The cells are washed twice by centrifugation and resuspension in PBS plus 0.05% sodium azide, pelleted and then resuspended in 50 μl Staining Solution (TdT enzyme/FITC-dUTP in cacodylate buffered saline). The cells are incubated at 37° C. for at least one hour. The staining is stopped by the addition of 1.0 ml PBS pus 0.05% sodium azide. The cells are pelleted by centrifugation at 300× g for 5 min, resuspended in PBS pus 0.05% sodium azide, and the repelleted. The supernatant is removed by aspiration and the pellet is incubated for 30 minutes at room temperature in the dark. The cells are analyzed by flow cytometry.

### D. Example 4

### Propidium Iodide Staining

Nine-day-adherent RA SF macrophages are combined with recombinant sFasL (1-50 ng/ml) and apoptosis is induced by adding sFasL cross-linker and incubating for 24 hours. Cultures are then harvested by 0.02% EDTA, fixing overnight in 70% ethanol, stained with propidium iodide (Roche Molecular Biochemicals, Indianapolis, Ind.), and the subdiploid peak, immediately next to the G₀/G₁ peak (2N), is determined by flow cytometry. It may be necessary to exclude objects with minimal light scatter, possibly representing debris, which would artificially increase the estimate of the subdiploid population.

### E. Example 5

### Anti-Histone Sandwich Assay

Nine-day-adherent RA SF macrophages are combined with recombinant sFasL (1-50 ng/ml) and apoptosis is induced by adding sFasL cross-linker and incubating for 24 hours. After the incubation, the cells are pelleted by centrifugation and the supernatant (containing DNA from necrotic cells that leaked through the membrane during incubation) is discarded. The cells are resuspended in Lysis Buffer and incubated 30 min at room temperature. After lysis, cell nuclei and unfragmented DNA are pelleted by centrifugation at 20 000× g for 10 min.

An aliquot of the supernatant (i.e., cytoplasmic fraction) is transferred to a well of a microtiter plate coated with anti-histone antibody. The complexes are bound to the plate via streptavidin-biotin-interaction. The immobilized antibody-DNA-antibody complexes are washed three times to remove any components that are not immunoreactive. The bound complexes are detected with anti-DNA (peroxidase-conjugated) monoclonal antibodies revealed by a peroxidase substrate and amount of colored product (and thus, of immobilized antibody-histone complexes) is determined spectrophotometrically. The quantitative colorimetric measurement of the DNA-histone complex is proportional to the total amount of apoptotic cells present in the cell population tested.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of alleviating at least one symptom of rheumatoid arthritis comprising cross-linking sFasL in a joint in a patient having rheumatoid arthritis.

2. The method of claim 1, wherein cross-linking sFasL increases macrophage apoptosis by at least 130%.

3. The method of claim 1, wherein the patient is resistant to methotrexate therapy.

4. The method of claim 1, wherein the patient is a TNF-α blockade nonresponder.

5. The method of claim 4, wherein the patient is a TNF-α blockade hyperplasia nonresponder, TNF-α blockade cartilage nonresponder, or a TNF-α blockade double nonresponder

6. The method of claim 1, wherein the symptom of rheumatoid arthritis is an abnormally increased synovial cell density.

7. The method of claim 1, wherein the symptom of rheumatoid arthritis is an abnormally high rate of cartilage degradation

8. The method of claim 1, wherein the symptom of rheumatoid arthritis is an abnormally high concentration of IL-6 in synovial tissue.

9. The method of claim 1, further comprising administering an anti-rheumatic drug to the patient.

10. The method of claim 9, wherein the anti-rheumatic drug is a symptom-relieving anti-rheumatic drug.

11. The method of claim 9, wherein the anti-rheumatic drug is a disease-modifying anti-rheumatic drug.

12. The method of claim 9, wherein the anti-rheumatic drug is an antagonist of FLIP activity to a patient having rheumatoid arthritis

13. The method of claim 12, wherein the antagonist of FLIP activity decreases FLIP activity by at least 25%.

14. The method of claim 13, wherein the antagonist of FLIP activity decreases FLIP activity by at least 75%.

15. The method of claim 14, wherein the antagonist of FLIP activity decreases FLIP activity by at least 95%.

16. The method of claim 12, wherein the antagonist of FLIP activity is a protein.

17. The method of claim 16, wherein the protein is oxidized low-density lipoprotein, ectopic-p53, IFN-β, PPAR ligand, EIA, or hemin.

18. The method of claim 12, wherein the antagonist of FLIP activity is a nucleic acid.

19. The method of claim 18, wherein the nucleic acid is an antisense inhibitor.

20. The method of claim 19, wherein the antisense inhibitor comprises the sequence, 5′-GACTTCAGCAGACATCCTAC-3′ (SEQ ID NO: 2).

21. The method of claim 12, wherein the antagonist of FLIP activity is a small molecule.

22. The method of claim 21, wherein the small molecule is selected from the group consisting of cyclohexamide, actinomycin D, 5-fluorouracil, doxorubicin, cisplatin, sodium butyrate, bisindolylmaleimides, H7, calphostin C, chelerythrine chloride, CDDO (triterpenoid 2-cyano-3,12-dioxoolean-1,9-dien-28-oic acid), and PS-341.

23. The method of claim 9, wherein the anti-rheumatic drug is selected from the group of methotrexate, a TNF-α antagonist, an interleukin-1 receptor antagonist and a steroid.

24. The method of claim 9, wherein the patient is a TNF-α blockade resistant patient and the anti-rheumatic drug is a TNF-α antagonist.

25. A method of manufacturing a drug for use in the treatment of rheumatoid arthritis comprising:
(a) identifying a compound as useful in the treatment of rheumatoid arthritis by:
(i) assaying the compound for the ability to cross-link sFasL and identifying the compound as a cross-linker;
(ii) comparing an amount of macrophage apoptosis in the presence of the cross-linker with an amount of macrophage apoptosis in the absence of the cross-linker; and
(iii) selecting the compound as useful in the treatment of rheumatoid arthritis when the amount of macrophage apoptosis in the presence of the cross-linker is greater than the amount of macrophage apoptosis in the absence of the cross-linker; and
(b) formulating said compound for human consumption.

26. The method of claim 25, wherein the compound is identified as useful in the treatment of rheumatoid arthritis when the amount of macrophage apoptosis in the presence of the compound is at least 2.3-fold the amount of macrophage apoptosis in the absence of the compound.

27. The method of claim 26, wherein the compound is identified as useful in the treatment of rheumatoid arthritis when the amount of macrophage apoptosis in the presence of the compound is at least 5-fold the amount of macrophage apoptosis in the absence of the compound.

28. The method of claim 27, wherein the amount of macrophage apoptosis is measured by a process comprising the steps of:
(1) exposing a population of cells to an inducer of apoptosis in the presence or absence of the compound; and
(2) measuring the percentage of cells in the population having DNA fragmentation wherein the percentage of cells having DNA fragmentation represents the amount of macrophage apoptosis.

29. The method of claim 28, wherein the inducer of apoptosis is soluble Fas ligand.

30. The method of claim 28, wherein the percentage of cells having DNA fragmentation is measured by FACS analysis of propidium uptake of cells.

31. The method of claim 28, wherein the percentage of cells having DNA fragmentation is measured by TUNEL assay.

32. The method of claim 25, wherein the amount of macrophage apoptosis is measured by a process comprising the steps of:
(1) exposing a population of cells to an inducer of apoptosis in the presence or absence of the compound; and
(2) measuring a percentage of cells in the population expressing phosphatidylserine on the extracellular surface of the cell membrane
wherein the percentage of cells expressing phosphatidylserine on the extracellular surface of the cell membrane represents the amount of macrophage apoptosis.

33. The method of claim 32, wherein the inducer of apoptosis is soluble Fas ligand.

34. The method of claim 32, wherein the percentage of cells expressing phosphatidylserine present on the extracellular surface of the cytoplasmic membrane is measured by binding of annexin V to the phosphatidylserine.

35. The method of claim 34, wherein the annexin V is conjugated to a fluorescent marker.

36. The method of claim 25, wherein the compound is a protein.

37. The method of claim 25,.wherein the compound is an antibody.

38. The method of claim 25, wherein the compound is a small molecule.

39. A method of identifying a compound useful in the treatment of rheumatoid arthritis, which method comprises:
(a) assaying the compound for the ability to cross-link sFasL and identifying the compound as a cross-linker;
(b) comparing an amount of macrophage apoptosis in the presence of the cross-linker with an amount of macrophage apoptosis in the absence of the cross-linker; and
(c) selecting the compound as useful in the treatment of rheumatoid arthritis when the amount of macrophage apoptosis in the presence of the cross-linker is greater than the amount of macrophage apoptosis in the absence of the cross-linker.

40. The method of claim 39 for screening a collection of compounds, further comprising repeating steps (a), (b), and (c) for each compound of the collection, wherein at least one compound of the collection is selected as useful for the treatment of rheumatoid arthritis.

41. A package comprising:
a) a compound capable of cross-linking soluble Fas ligand; and
b) a label with instructions for administering the compound for treating rheumatoid arthritis.

42. A pharmaceutical composition for the treatment of rheumatoid arthritis comprising:
a) a compound capable of cross-linking soluble Fas ligand; and
b) a pharmaceutical excipient.


SEQUENCE LISTING (2 sequences)

SEQ ID NO: 1 - protein, 15 aa, Homo sapiens
SLEKQIGHPSPPPEK

SEQ ID NO: 2 - DNA, 20 bp, Homo sapiens
gacttcagcagacatcctac